(19) United States
(12) Patent Application Publication
(10) Pub. No.: US 2006/0161199 A1
(43) Pub. Date: Jul. 20, 2006

(54) Systems and methods for filling spaces within the body using asymmetrically strained filaments

(75) Inventor: Dale R. Peterson, La Jolla, CA (US)

Correspondence Address: RYAN KROMHOLZ & MANION, S.C., POST OFFICE BOX 26618, MILWAUKEE, WI, 53226, US

(73) Assignee: The Vertical Group, L.P.

(21) Appl. No.: 11/331,952

(22) Filed: Jan. 13, 2006

Related U.S. Application Data
(60) Provisional application No. 60/644,376, filed on Jan. 14, 2005.

(51) Int. Cl. A61M 29/00

(52) U.S. Cl. 606/200

(57) ABSTRACT

The application of asymmetric strain to a filament transforms the filament into curled or complex three-dimensional shape suited for packing an aneurysm.

### RELATED APPLICATION

This application claims the benefit of U.S. Provisional Patent Application Ser. No. 60/644,376, filed 14 Jan. 2005, and entitled “Systems and Methods for Treating Aneurysms Using Asymmetrically Strained Filaments.”

### FIELD OF THE INVENTION

This invention relates generally to systems and methods for filling spaces or volumes in need of healing or repair with an animal body, e.g., for treating an aneurysm.

### BACKGROUND OF THE INVENTION

Spaces or volumes can develop within an animal body that need healing or repair.

For example, aneurysms are a life-threatening vascular defect in which the wall of a blood vessel has weakened and ballooned. Although they can occur anywhere in the body, aneurysms are particularly dangerous if they rupture in the brain or along a major artery such as the aorta. Cerebral aneurysms are usually treated via three popular methods: surgery, filled with a polymer solution which solidifies in situ, or packed with aneurysm coils. Surgery is difficult and dangerous in the brain and so most cerebral aneurysms are treated by interventional methods.

Many interventional techniques have been tried in the past. Microfibrillar collagen injected into a lumen quickly embolized, but was not often permanent. Balloons have been inflated with resin which solidifies, but occasionally the aneurysms burst because of friction of the balloon on the aneurysm walls or the balloon was overfilled. It was difficult to control the distribution of injected polymer beads used to embolize the vessels with the aneurysm. Solutions of polyvinyl alcohol that solidify into a foam are being tested currently as is cyanoacrylate cement. These two approaches are fast, but it is difficult to control where the material hardens. The most common approach by far is the use of metal coils.

Today the most popular coils are made of platinum in various sizes and gauges. Aneurysms are gently filled with large coils and then packed with small coils in a procedure that may require a few hours. Coils are also made of stainless steel, tungsten, and gold. They are termed Guglielmi detachable coils (GDC) after the inventor of the detachment method (U.S. Pat No. 5,122,136 and 5,354,295)—they are released from the wires used to push them into position through catheters via an electrochemically erodable link.

Most of the coils in use are helical spirals, but a variety of other shapes have been described and patented. U.S. Pat. No. 5,690,666 describes limp coils, chains, or braids which assume random shapes when delivered into an aneurysm. Several other patents describe coils which adopt a secondary structure when released into an aneurysm such as U.S. Pat. No. 5,645,558—spheres; U.S. Pat. No. 5,649,949—conicals; U.S. Pat. No. 5,639,277—multiaxial figures; and U.S. Pat. No. 5,645,082—random mass.

Several detachment methods have been described in addition to electrolytic release. U.S. Pat. No. 5,618,711 describes hydraulic delivery of individual coils through a catheter. Several different mechanical detachment methods have been patented such as U.S. Pat. No. 5,234,437 in which the push wire is threaded and unscrewed from the coil; U.S. Pat. No. 5,250,071 in which the coil and push wire have interlocking clasps; and U.S. Pat. No. 5,304,195 and 5,261,916 which have a ball on the push wire that interlocks with the coil until pushed clear of the catheter. U.S. Pat. No. 5,494,884 discusses the use of a link between the push wire and the coil which is dissolvable by a fluid. U.S. Pat. No. 6,312,421 discloses coils made of hydrogels which are cut to length. U.S. Pat. No. 6,478,773 claims detachment of aneurysm coils by melting a linking fiber.

Although widely used, GDC coils have some serious shortcomings. Coils must be fitted into the aneurysm and detached one at a time, a process that can take hours. U.S. Pat. No. 6,551,305 describes a method for delivering and detaching multiple, sequential coils, but the coils are still of discrete size and shape.

More troubling are the well-filled and embolized aneurysms that re-canalize when the clot dissolves and the healing process is complete. About ⅓ of all aneurysms require further treatment because of re-canalization including approximately 10% of those initially well-filled and embolized. Several approaches have been tried to reduce the rate of re-canalization including packing the aneurysm more densely with coils, attaching fine polymer fibers to standard coils; coating coils with polymers or biopolymers (U.S. Pat. No. 6,231,590, and 6,187,024); using coils of polymers that promote better healing; adding growth factors such as basic fibroblast growth factor or vascular endothelial growth factor to the coils to promote fibrosis; and coating coils with cells which promote fibrosis (Marx, et al, AJNR 22: 323-333, 2001). Coils mixed with or coated with polymers are being used, but have inferior handling and packing properties. Polymer coils have not been adopted because of their poor handling and packing. Polymer coils packed into a delivery cannula also lose much of their ability to rebound to complex shapes that permit efficient packing.

### SUMMARY OF THE INVENTION

The invention provides systems and methods that greatly improve and simplify the procedure for filling spaces or volumes within an animal body in need of healing or repair, e.g., for treating aneurysms, by providing a scaffold for repair cells or tissue. The systems and methods operate in a minimally invasive manner.

The systems and methods apply asymmetric strain to a filament to transform the filament into curled or complex shapes, e.g., suited for packing an aneurysm. The filament provides a scaffold for repair cells or tissue, which can be introduced with the filament, after the filament is introduced, or supplied naturally by the body. The systems and methods apply asymmetric strain to the filament shortly before or during the act of conveying the filament to the space or volume, such as an aneurysm. The asymmetrically strained filament can be cut to length in situ with energy or mechanical force to expedite the packing procedure. The space or volume, such as an aneurysm, can be packed with continuous filaments rather than placing and detaching individual coils of discrete lengths.

The packing efficiency can be increased by varying the amount and direction of asymmetric strain along the length of the filament so that it curls into complex shapes once outside the delivery catheter.

Asymmetric strain can be applied in various ways. In one arrangement, the filament is fed between one or more rollers as the filament is fed through the delivery catheter. The rollers can include a surface pattern that crimps the filament. Also, the rollers can turn at different rates. In another arrangement, the filaments are forced over a sharp lip with a small radius of curvature. In another arrangement, the filaments are treated asymmetrically by heating or cooling.

The application of asymmetric strain to a filament before or in the act of filling a space or volume, such as an aneurysm, makes possible the use of materials which prompt more durable healing of the space or volume, such as an aneurysm, but are unable, without requisite asymmetric straining, to form dense, complex, three-dimensional shapes after packaging and storage for long time periods. Polyhydroxyalkanoates such as poly-4-hydroxybutyrate are excellent candidates.

Manufacturing the filament with a surface crimp pattern can reduce the amount of stress needed to produce the desired curl of the filament. The crimp pattern can be applied during fabrication of the filament or as a subsequent coating with the same or dissimilar materials.

### BRIEF DESCRIPTION OF THE DRAWINGS

FIG. 1 is view of a device that, in use, treats a space or volume within an animal body, such as an aneurysm.

FIGS. 2 to 4 show the use of the device shown in FIG. 1 to pack an aneurysm with one or more filaments that have been asymmetrically strained to form complex three-dimensional packing shapes.

FIG. 5 is a largely diagrammatic view of a device like that shown in FIG. 1, which includes a forming mechanism that, in use, receives filament in linear form from a source and transforms it by asymmetric straining into a filament that, when deployed from a catheter body, curls into a complex three-dimensional packing shape, like that shown in FIGS. 3 and 4.

FIG. 6 is a view of an embodiment of a forming mechanism of the type shown in FIG. 5.

FIG. 7 is a view of another embodiment of a forming mechanism of the type shown in FIG. 5.

FIG. 8 is a view of an embodiment of a forming mechanism of the type shown in FIG. 5.

### DESCRIPTION OF PREFERRED EMBODIMENTS

Although the disclosure hereof is detailed and exact to enable those skilled in the art to practice the invention, the physical embodiments herein disclosed merely exemplify the invention which may be embodied in other specific structures. While the preferred embodiment has been described, the details may be changed without departing from the technical features described below.

A. Overview

FIG. 1 shows a device 10 for treating a space or volume within an animal body. The device 10 comprises a flexible catheter body 12 carried by a handle 14. The catheter body 12 may be constructed, for example, by extrusion using standard flexible, medical grade plastic materials. The handle 14 may be constructed, for example, from molded plastic. The handle 14 is sized to be conveniently held by a clinician, to introduce the catheter body 12 into an interior body region where a space or volume in need of healing or repair exists.

The space or volume in need of healing or repair can take various forms. Since the device 10 is well suited for the treatment of an aneurysm, its use for this purpose will be described. However, it will be appreciated that the technical features of the device 10 as will be described are well suited for use for treating any space or volume within an animal body in need of healing or repair.

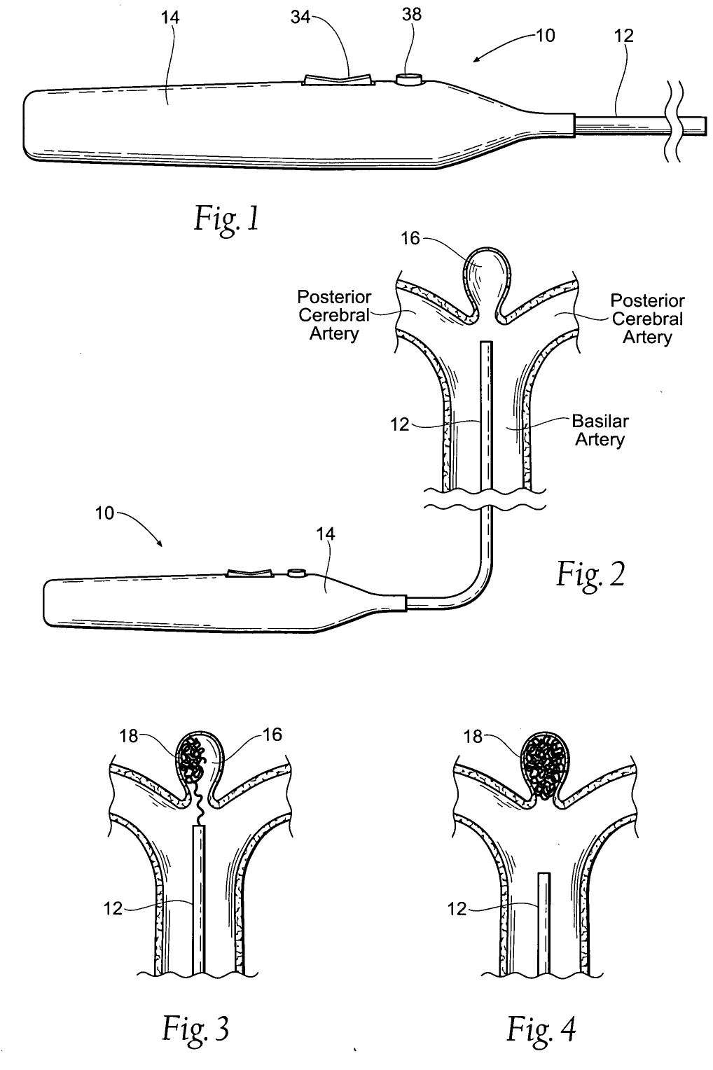

FIG. 2 shows the flexible catheter body 12 deployed, for the purpose of illustration, in the region of a Berry aneurysm 16 at the bifurcation of the basilar artery into the two posterior cerebral arteries. A Berry aneurysm of the type shown is a balloon-like sac that forms on the weak part of the wall of an artery in vessels of or near the cerebral arterial circle and the medium-sized arteries at the base of the brain.

The device 10 is manipulated in conventional fashion to deploy the catheter body 12 by intra-vascular approach to the region of the aneurysm 16. In use (as FIG. 3 shows), the catheter tube 12 delivers into the aneurysm 16 one or more filaments 18. The filaments are subject to asymmetric straining prior to or in the act of delivery so that, when released into the aneurysm from the constraints of the catheter tube 12, the filaments curl tightly into complex, three-dimensional packing shapes within the aneurysm. The release of the one or more filaments 18 serve to fill or pack the aneurysm to embolize it, as FIG. 4 shows. The filaments provide a scaffold for repair cells and tissue.

B. The Forming Mechanism

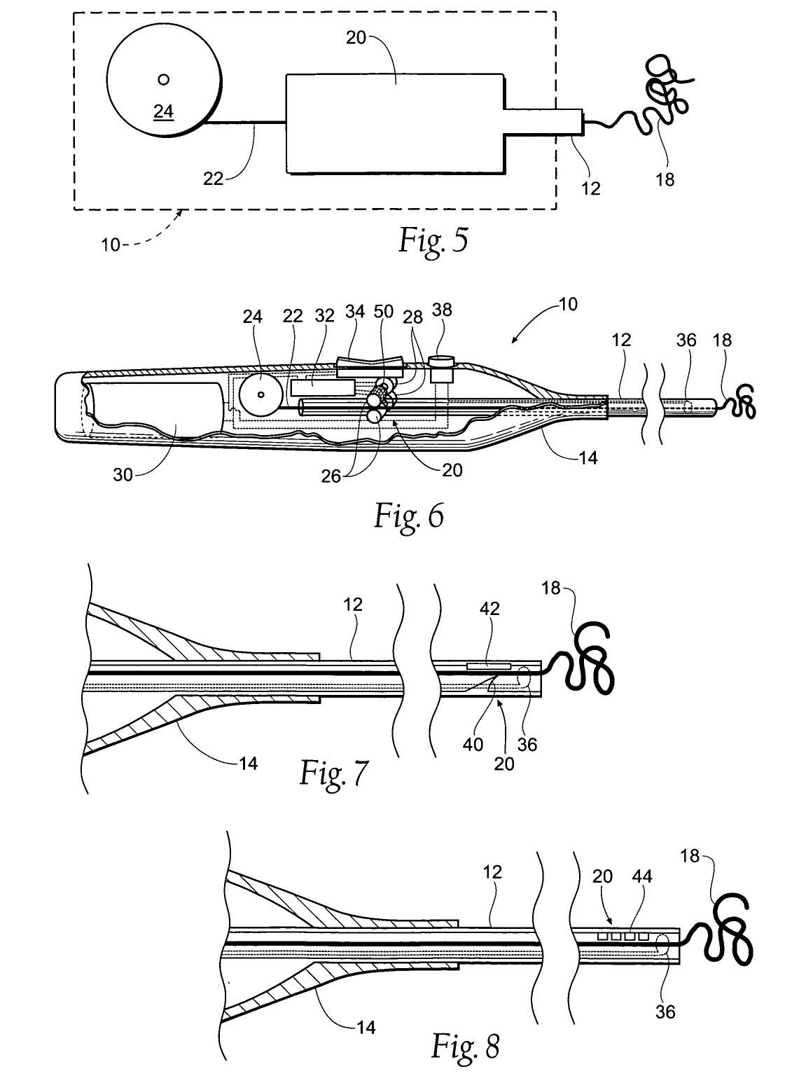

As FIG. 5 generally shows, the device 10 includes a forming mechanism 20 that receives filament 22 in linear form from a source 24 and transforms it by asymmetric straining into a filament 18 that, when deployed from the catheter body 12, curls into a complex three-dimensional packing shape. Desirably, the forming mechanism 20 is sized and configured to be carried within the handle 14 (as FIG. 6 shows), or within the catheter body 12 (as FIG. 7 shows), or both. Desirably, as FIG. 6 shows, the source 24 of linear filament 22 is also sized and configured to be carried within the handle 14.

The forming mechanism 20 applies asymmetric strain to the linear filament 22 before it is placed into the aneurysm. The asymmetric strain induces the linear filament 22 to curl tightly when released into the aneurysm, forming the filament 18 having a curled or complex three-dimensional packing shape.

The packing efficiency of the filament 18 can be further increased, e.g., by varying the amount and/or orientation of the asymmetric strain along the filament 22. As a result, when deployed within the aneurysm sac, the filament 18 produces a dense three-dimensional mat which fills with clotted blood, and later with fibrous tissue, to seal off the aneurysm from blood flow and pressure.
1. Asymmetric Rolling

The forming mechanism 20 can be variously configured to create asymmetric strain.

For example, as FIG. 6 shows, the forming mechanism 20 can include two or more rollers 26 mounted within the housing 14, between which the linear filament 22 is fed from a source 24. In FIG. 6, the source 24 is shown to be a spool that is also mounted within the handle 14.

The rollers 26 are powered by motors 28 coupled to an on-board power source 30, e.g., a battery. A control switch 34 on the handle 14 can be manipulated by the clinician to turn the motors 28 on and off. One or more rollers 26 can be provided with a surface pattern 50 that selectively crimps the filament 18 as it traverses the rollers to create asymmetric strain.

As shown in FIG. 6, the motors 28 are coupled to an on-board motor controller 32, which is desirably programmable. In this arrangement, the controller 32 can command the rollers 26 to rotate at different rates. In this way, asymmetric strain can be applied to the linear filament 22 as it is conveyed through the rollers 26 into the catheter tube 12. When discharged from the constraints of the catheter tube 12, the asymmetrically strained filament 18 curls into its desired complex three-dimensional packing shape. One or more of the rollers 26 can include a surface pattern 50 to crimp the filament 18, to provide addition asymmetric strain, or the rollers 26 can be free of a surface pattern.

The asymmetrically strained filament is desirably selectively cut to length in situ, e.g., with a cutter element 36 mounted at or near the distal end of the catheter tube 12. The cutter element 36 can be operated mechanically to sever the filament, or by heat to melt the filament. A control switch 38 is desirably mounted on the handle 14 to selectively actuate the cutter element 36.

The aneurysm can be filled with continuous filaments rather than placing and detaching individual coils of discrete lengths.

To enhance the packing density, the controller 32 can, e.g., be programmed to vary the strain rate to enhance the packing density. For example, the controller 32 can vary the differential in rotational rates of the rollers 26 over time. Alternatively, or in combination, the orientation of one or more the rollers 26 relative to filament can be made adjustable under the control of the controller 32, to thereby vary the axis of the applied strain.
2. Asymmetric Dragging

As FIG. 7 shows, the forming mechanism 20 can include a sharp lip 40 located either within the handle 14 or (as FIG. 7 shows) within the catheter tube 12. A suitable conveying mechanism (e.g., rollers as shown in FIG. 6) conveys the linear filament 22 in a path over the lip 40. The conveying mechanism can be placed either before or after the lip 40, depending upon the stiffness of the filament 22. A filament 22 lacking the requisite stiffness to be pushed across the lip 40 (i.e., using a conveying mechanism located before the lip) will need to be pulled across the lip 40 (i.e., using a conveying mechanism located after the lip).

The lip 40 applies asymmetric strain to one side of the filament as it is conveyed over the lip 40 and through the catheter tube 12. When discharged from the constraints of the catheter tube 12, the asymmetrically strained filament 18 curls into its desired complex three dimensional packing shape.

In this arrangement, the packing density can be enhanced or controlled, e.g., by use of a platen 42 that varies the force on the filament as it passes over the lip 40, or by varying the axis of the filament relative to the lip 40.

As before described, the asymmetrically strained filament 18 can desirably be selectively cut to length in situ, e.g., with a cutter element 36 mounted distal to the lip 40, at or near the distal end of the catheter tube 12 or by simply pulling the filament back towards the spool briefly so that the filament is cut by the lip 40. The asymmetric application of strain by the lip 40 can be accomplished alone, or in combination by the asymmetric strain applied by the rollers 26 or another type of asymmetric forming mechanism.
3. Asymmetric Heating/Cooling

As FIG. 8 shows, the forming mechanism 20 can include differential heating or cooling elements 44 located either within the handle 14 or (as FIG. 8 shows) within the catheter tube 12. A suitable conveying mechanism (e.g., rollers as shown in FIG. 6) convey the linear filament 22 in a path adjacent the heating or cooling elements 44. The heating or cooling elements 44 applies asymmetric strain to one side of the filament as it is conveyed past the heating or cooling elements 44 and through the catheter tube 12. When discharged from the constraints of the catheter tube 12, the asymmetrically strained filament 18 curls into its desired complex three-dimensional packing shape 18.

The asymmetrically strained filament can desirably be selectively cut to length in situ, e.g., with a cutter element 36 mounted distal to the heating or cooling elements 44 at or near the distal end of the catheter tube 12 or simply by increasing the heat from heating elements 44.

The asymmetric application of strain by the heating or cooling element 44 can be accomplished alone, or in combination by the asymmetric strain applied by the rollers 26, and/or lip 40, and/or another type of asymmetric forming mechanism.

In this arrangement, the packing density can be enhanced and controlled, e.g., by varying the amount of heat applied or removed, the rate at which heat is transferred, and varying the surface of the filament which is thermally treated.

A given forming mechanism 20, or a given combination of forming mechanisms 20 may be located at the end, middle, beginning, or prior to entering the device 10. Multiple modes may be used to induce asymmetric strain (e.g., rollers and heat), multiple sources can be used (e.g., several lips), and the asymmetric strain can be applied at multiple locations (e.g. prior to the delivery catheter and near its exit). The packing density may be further enhanced by using filaments of different diameters, of different materials, or with different coatings.

The force required to create asymmetric strain can be reduced by using filaments with a crimped surface, by applying a crimped surface coating, or by applying a coating material to the filaments that has a low yield strength. Such coatings can be the same or dissimilar materials. Filaments with a crimped surface can be manufactured by applying vibration to the spinning head, varying the spinning take-up rate or location, or passing the filament across texturing rolls, for instance.

C. The Filament

The linear filament 22 provided by the source 24 can be made of many different biocompatible materials, e.g., metals, synthetic polymers, resorbable polymers, natural polymers, glasses, ceramics, and combinations of the above.

Metal materials include nitinol, platinum, platinum-tungsten alloys, and stainless steel.

The on site asymmetric treatment of a linear filament 22 makes possible the use of polymeric materials that can induce better healing of the aneurysm, but which could not, without asymmetric straining, be fabricated into appropriate shapes. Polymer materials that can be symmetrically strained on site include polyesters, polyalkenes, polyurethanes, polyamides, polyacrylates, polyhydroxyalkanoates, polydioxanone, polylactide, polyglycolide, polycaprolactone, trimethylenecarbonate, proteins, polysaccharides, and polyaminoglycans. The response these materials have to the application of on site asymmetric straining makes possible their use for densely packing aneurysms. Importantly, by using the forming mechanism 20, materials which prompt more durable healing of the aneurysm, but which are otherwise unable to form dense, complex, three-dimensional shapes after packaging and storage for long time periods, can now be used. Polyhydroxyalkanoates such as poly-4-hydroxybutyrate are excellent candidates.

Biologics may be added to the filament or filaments 18 selected for use, to further improve their ability to heal aneurysms. The biologics may be incorporated into the filament or filaments 18 or applied to the surface as a coating. Suitable biologics include drugs, growth factors, peptides, transcription factors, nucleic acids or analogs, and cells. Growth factors such as fibroblast growth factor, vascular endothelial growth factor, transforming growth factor, or their mimetics are particularly promising. These materials can be introduced on the filament, or with the filament, or after the filament. Tissue supplied naturally by the body can also interact with the filament 18 to provide repair cells or tissue growth.

Interventionalists prefer to use packing materials that are radio-opaque, so they can be visualized during the procedure. Polymeric filaments subject to asymmetric straining can be made radio-opaque by filling them with metal particles (such as tungsten, gold, platinum, tantalum), or contrast agents (such as hypaque or barium sulfate), or by coextruding the filaments with metal wires.

The foregoing is considered as illustrative only of the principles of the invention. It should be appreciated that the device, systems, and methods as described incorporate many technical features, which include:

1. The application of asymmetric strain to a filament to transform the filament into curled or complex shapes suited for packing a space or volume within an animal body, including but not limited to an aneurysm.

2. The application of asymmetric strain to the filament shortly before or during the act of conveying the filament to a space or volume within an animal body, including but not limited to an aneurysm.

3. The cutting of an asymmetrically strained filament to length in situ with energy or mechanical force to expedite the packing procedure.

4. The packing of an aneurysm with continuous asymmetrically strained filaments, instead of placing and detaching individual coils of discrete lengths.

5. Increasing the packing efficiency of asymmetrically strained filaments by varying the amount and direction of asymmetric strain along the length of the filament so that it curls into complex three dimensional packing shapes once outside the delivery catheter.

6. Asymmetrically straining a filament as the filament is conveyed through the delivery catheter by feeding filament between two rollers, which grip the filaments tightly and turn at different rates and one or more of which, separately or in combination, can include a surface pattern to crimp the filament.

7. Asymmetrically straining a filament as the filament is conveyed through the delivery catheter by forcing the filament over a lip with a small radius of curvature.

8. Asymmetrically straining a filament as the filament is conveyed through the delivery catheter by treatment employing heating or cooling.

9. The act of filling an aneurysm using a filament material which prompts durable healing of the aneurysm, e.g. polyhydroxyalkanoates such as poly-4-hydroxybutyrate, by asymmetrically straining the filament material to form dense, complex, three-dimensional packing shapes.

10. Manufacturing a filament with a surface crimp pattern to reduce the amount of stress needed to produce the desired curl of the filament as a result of asymmetric straining. The crimp pattern can be applied during fabrication of the filament or as a subsequent coating with the same or dissimilar materials.

Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

I claim:

1. A device for filling a volume within an animal body comprising
a dispensing body,
a source of a filament in linear form, and
a forming mechanism that receives the filament from the source and dispenses the filament from the dispensing body into the volume, the forming mechanism being sized and configured to apply an asymmetric strain to the filament so that, when dispensed from the dispensing body, the filament curls into a three-dimensional packing shape.

2. A device according to claim 1
wherein the forming mechanism includes one or more rollers having a surface pattern that crimps the filament.

3. A device according to claim 1
wherein the forming mechanism includes two or more rollers driven at different rates of rotation.

4. A device according to claim 1
wherein the forming mechanism includes a lip over which the filament is forced.

5. A device according to claim 1
wherein the forming mechanism includes means for heating or cooling the filament.

6. A device according to claim 1
further including a component sized and configured to cut the filament to a desired length.

7. A device according to claim 1
wherein the forming mechanism is sized and configured to vary the asymmetric strain along the filament.

8. A method of treating a volume in an animal body in need of healing or repair comprising
introducing a filament into the volume to provide a scaffold for repair cells or tissue, and
while introducing the filament, applying an asymmetric strain to the filament so that the filament curls into a three-dimensional packing shape within the volume.

9. A method according to claim 8
wherein the volume comprises an aneurysm.
